(19) United States
(12) Patent Application Publication
(10) Pub. No.: US 2015/0073000 A1
(43) Pub. Date: Mar. 12, 2015

(54) STABLE READY-TO-USE PHARMACEUTICAL COMPOSITION OF PEMETREXED

(71) Applicant: Fresenius Kabi Oncology Limited, New Delhi (IN)

(72) Inventors: Dhiraj Khattar, Gurgaon (IN); Rajesh Khanna, Gurgaon (IN); Mukti Yadav, Gurgaon (IN); Krishanu Burman, Gurgaon (IN)

(21) Appl. No.: 14/387,670

(22) PCT Filed: Mar. 25, 2013
(86) PCT No.: PCT/IB2013/052356

§ 371 Date: Sep. 24, 2014

(30) Foreign Application Priority Data
Mar. 27, 2012 (IN) 912/DEL/2012

(51) Int. Cl. A61K 31/519; A61J 1/14

(52) U.S. Cl. CPC A61K 31/519; A61J 1/14
USPC 514/265.1; 544/280; 206/213.1

(57) ABSTRACT

A stable ready-to-use pharmaceutical composition comprising pemetrexed or pharmaceutically acceptable salts thereof, wherein the composition is free from antioxidants, amino acids and chelating agents. Also provided is a process for preparing a stable ready-to-use pharmaceutical composition comprising the steps: i) purging inert gas into a parenterally acceptable aqueous solvent until the dissolved oxygen content of the solvent comes to less than 7 mg/L, preferably less than 3 mg/L; ii) adding pemetrexed disodium under stirring; iii) adjusting the pH of the resulting solution to between 4 to 9; iv) optionally adding additional aqueous solvent; wherein the composition is purged with inert gas throughout the entire process.

### FIELD OF THE INVENTION

The present invention relates to a ready-to-use pharmaceutical composition comprising the known compound Pemetrexed that is free from antioxidants, amino acids and chelating agents; which liquid composition is stable and pharmaceutically elegant.

### BACKGROUND OF THE INVENTION

Certain folic acid antimetabolites are known to be antineoplastic agents. These compounds inhibit enzymatic conversion involving metabolic derivatives of folic acid. One such compound described by U.S. Pat. No. 5,344,932, known as “Pemetrexed” represented by Formula-I shown below, is currently formulated into a concentrated liquid for administration as an infusion dosage form. This member of the folic acid family has been approved for treatment of malignant pleural mesothelioma and for second-line treatment of non small cell lung cancer. Pemetrexed disodium salt heptahydrate represented by Formula-II is marketed by Eli Lilly and Company under the trade name ALIMTA® as a sterile lyophilized powder for intravenous administration. The commercial product is reported to be a lyophilized powder of heptahydrate Pemetrexed disodium and mannitol. The lyophilized product is available in strengths of 100 mg/vial and 500 mg/vial and is reconstituted with 0.9% sodium chloride at a concentration of 25 mg/mL before its administration.

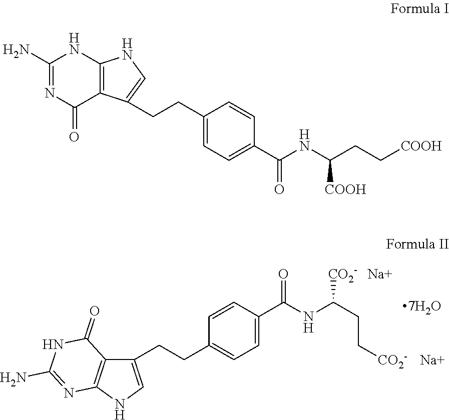

The formulation teachings of U.S. Pat. No. 5,344,932 provides that the compounds claimed therein can be administered parenterally.

A ready-to-use, stable, ready to reconstitute solution that could be stored at room temperature is particularly desired for a pharmaceutical such as Pemetrexed, wherein such ready-to-use pharmaceutical composition provides easier, safer handling, storage, and distribution. It is particularly desirable if the stable pharmaceutical composition can be prepared without the use of freeze drying techniques. The disadvantage of lyophilized drugs is that they have to be reconstituted, usually by injecting diluent through the septum into the vial. The drug is drawn up into a new syringe, the needle changed before finally being injected into the patient. The multiple steps make it inconvenient for use and provide an opportunity for injury from exposed needles. The desired liquid formulation can offer enhanced safety for caregiver handling of the cytotoxic materials. Further, a stable, ready-to-use pharmaceutical composition is more acceptable to the customer.

It was discovered that a simple, isotonic saline solution of Pemetrexed is not pharmaceutically acceptable for commercial purposes due to degradation of the solution to form unacceptable related substances. The chemical instability of Pemetrexed is mainly attributed to their oxidative degradation. Hence, the main challenge lies in formulating a stable pharmaceutical composition of Pemetrexed that has the minimum concentration of oxidative degradation impurities. All the prior arts mainly provide solutions to the problem related to oxidative degradation of the drug by using an antioxidant or an amino acid or a chelating agent in their pharmaceutical composition.
1. Bernd et al in U.S. Pat. No. 6,686,365 discloses a stable ready-to-use (RTU) formulation of Pemetrexed which is developed by using antioxidants/amino acids like L-Cysteine, Monothioglycerol and Thioglycolic acid. Hence, use of an antioxidant in the formulation is the key element in developing a stable pharmaceutical composition of Pemetrexed.
2. Yanling et al in CN Patent No. 101081305, again discloses a RTU formulation of Pemetrexed stabilized by using antioxidant like L-arginine, L-glutathione, L-methionine and L-tryptophan. Hence, use of an antioxidant in the formulation is the key element in developing a stable pharmaceutical composition of Pemetrexed.
3. Palepu et al in PCT Application No. WO 2012/015810, again claims a RTU solution formulation of Pemetrexed along with an antioxidant, a chelating agent and dissolved in a pharmaceutically acceptable fluid. Hence, use of antioxidants and chelating agents in the formulation is the key element in developing a stable pharmaceutical composition of Pemetrexed.

Hence, from all the above mentioned prior art disclosures it is evident that the key element in all these pharmaceutical compositions of Pemetrexed is that either antioxidants or amino acids or chelating agents are invariably present in all these formulations. The main role of these antioxidants or amino acids or chelating agents is to prevent the oxidative degradation of Pemetrexed and provide chemical stability to the various parenteral formulations. This clearly indicates that till date a stable liquid pharmaceutical composition of Pemetrexed has always been obtained by the addition of antioxidants, amino acids and chelating agents in the formulation. Hence, the presence of antioxidants, amino acids and chelating agents in the dosage form has been found to be the quintessential element in the formulation of a stable pharmaceutical composition of Pemetrexed as it reduces the oxidative degradation of the drug and provides stability to the formulation.

However, a point of mention is that such agents as antioxidants, amino acids and chelating agents qualify as extraneous agents in the formulation of any pharmaceutical compositions. It may further be mentioned that Health Authorities all over the world are very concerned about the level of such extraneous agents as antioxidants, amino acids and chelating agents in the pharmaceutical compositions. As a consequence, regulatory approval norms today are very stringent about the nature and level of extraneous agents present in any drug product. In view of this, the range or freedom available to experiment with various extraneous agents such as antioxidants, amino acids and chelating agents is minimum and they cannot be utilized beyond a limited amount. The presence of any unapproved range of excipients in the pharmaceutical formulations may have harmful effects on the patients and hence, such formulations are not acceptable to the Health Authorities, even if such formulations are stable. Keeping the aforementioned limitations in mind it is essential for the formulators to develop a pharmaceutical composition that is stable and contains any extraneous agents in the formulation in quantities that fall within the regulatory limits.

Hence, there is a need to develop pharmaceutical compositions of Pemetrexed that are both stable as well as free of any extraneous agents such as antioxidants/amino acids/chelating agents and hence, such formulations are more patient compliant.

Against this backdrop the inventors of the present Application have surprisingly found that stable ready-to-use pharmaceutical compositions of Pemetrexed may be developed without the use of extraneous agents such as antioxidants, amino acids and chelating agents in the formulation that shows comparable stability to the currently marketed ALIMTA® lyophilized formulation and this forms the basis of the present Application.

### SUMMARY OF THE INVENTION

The present invention provides stable ready-to-use pharmaceutical compositions of Pemetrexed that is free from antioxidants, amino acids and chelating agents and shows comparable stability to the marketed formulation.

The most important aspect of the present invention is to provide stable ready-to-use pharmaceutical compositions of Pemetrexed that may be suitable for parenteral administration. Such stable ready-to-use formulation of Pemetrexed can be developed without the use of antioxidants or amino acids or chelating agents, by controlling the oxygen content of drug solution and vial headspace with the use of an inert gas viz nitrogen. Such parenteral formulations do not contain antioxidants, amino acids or chelating agents in their formulation, however they exhibit comparable stability profile to the currently marketed ALIMTA® lyophilized formulation and found to more stable in comparison to the Pharmaceutical composition using Antioxidants in place of Nitrogen under the same storage conditions.

### DETAILED DESCRIPTION OF THE INVENTION

The present invention is directed to stable ready-to-use pharmaceutical compositions of Pemetrexed that is free from antioxidants, amino acids and chelating agents and shows comparable stability to the currently marketed formulation.

The formulations as developed by the Inventors of the present Application are suitable for parenteral administration. Such stable ready-to-use formulation of Pemetrexed can be developed without the use of antioxidants, amino acids and chelating agents, by controlling the oxygen content of drug solution and vial headspace with the use of an inert gas viz nitrogen. Such parenteral formulations do not contain antioxidants, amino acids and chelating agents in their formulation; however they exhibit comparable stability to the currently available lyophilized marketed formulation of Pemetrexed.

These formulations are presented as a single vial presentation having Pemetrexed concentrations in the range of 2.5 to 50 mg/ml, of which the preferred concentration is 25 mg/ml. These pharmaceutical compositions are then administered via intravenous infusion to treat patients suffering from malignant pleural mesothelioma and for second-line treatment of non small cell lung cancer which is the approved indication of Pemetrexed.

As used herein, the term “pemetrexed” refers to the stable salts, acids and free base forms thereof. The term includes, for example, the free acid, the pharmaceutically acceptable alkali metal, alkaline earth metal, non-toxic metal, ammonium, and substituted ammonium salts, such as for example, the sodium, potassium, lithium, calcium, magnesium, aluminum, zinc, ammonium, trimethylammonium, triethylammonium, monoethanolammonium, triethanolammonium, pyridinium, substituted pyridinium, and the like.

The stable ready-to-use pharmaceutical composition of Pemetrexed is usually solvated in an aqueous solvent comprising water for injection.

In one embodiment of the present invention, the ready-to-use pharmaceutical composition of Pemetrexed has a pH between about 4 and about 9, preferably between about 5 and about 8 and more preferably in the range of about 6.6 and about 7.8. The pH of such ready-to-use pharmaceutical compositions of Pemetrexed may be adjusted with a pharmacologically acceptable pH adjusting agent such as an acid, base, buffer or their combination thereof. In an embodiment of the present invention the pH adjusting agent is hydrochloric acid or sodium hydroxide, or their combination thereof. The hydrochloric acid or sodium hydroxide may be in any suitable form, such as a 1N solution.

In another embodiment of the present invention, so as to minimize oxidation of the sensitive material it is also desirable to remove headspace oxygen and moisture or both from the sealable vessel as quickly as possible. This may be aided by, for example, purging the sealable container with a gas which is substantially oxygen-free, or substantially moisture free, or substantially oxygen and moisture free before, during or after step, or any combination thereof. Purging can be expected to reduce the oxygen level in the sealable container to a level of from about 0.5% to about 10%, typically about 5% or lower, depending on the efficiency of flushing and how quickly the container is sealed after flushing.

The gas used for purging the sealable container may be any appropriate inert gas known to those in the art, the most commonly used gases being argon, helium or nitrogen, or mixtures thereof. However the most preferred inert gas is nitrogen.

In another embodiment of the present invention, so as to increase the storage stability of the aqueous parenteral preparation, optionally it may be desirable to add antimicrobial agent to inhibit the growth of microbial organism which may occur accidently and contaminate the product during use. The antimicrobial agents selected are stable and effective in the parenteral formulations, the most commonly used being benzalkonium chloride, benzyl alcohol, phenol, chlorocresol, phenylmercuric salts, methylhydroxybenzoate, propylhydroxybenzoate. However the most preferred antimicrobial agents are methylhydroxybenzoate, propylhydroxybenzoate and benzalkonium chloride.

The invention is further illustrated by way of the following example, which in no way should be construed as limiting the scope of the invention.

### EXAMPLES

Various embodiments of the pharmaceutical formulations according to the present invention were prepared and studied for their stability and impurity profile when stored under accelerated stability conditions, which are illustrated below:

### Example 1

The pharmaceutical composition as provided in this example is a Stable ready-to-use pharmaceutical composition of Pemetrexed that is free from antioxidants, amino acids and chelating agents.

The ready-to-use pharmaceutical composition of Pemetrexed comprises of Pemetrexed disodium as the active ingredient, wherein Pemetrexed disodium was prepared from Pemetrexed diacid by taking suitable quantity of water for injection in a manufacturing vessel. Nitrogen was purged into water for injection until dissolved oxygen content of water for injection comes to less than 7 mg/L, preferably less than 3 mg/L. Pemetrexed Diacid was then added in water for injection to make a slurry. Fixed quantity of sodium hydroxide (4.7 mg/mL) in the form of 10% w/v solution was added to dissolve Pemetrexed Diacid. The pH was adjusted to 6.6-7.8 with either 10% w/v sodium hydroxide solution or 1N hydrochloric acid solution. Nitrogen purging was continued throughout the entire procedure. Final volume was made upto 100% with water for injection and drug solution was then filtered through a suitable 0.2μ filter. The filtered solution was then filled into vials and vials headspace was blanketed with nitrogen to achieve headspace oxygen content less than 8%, preferably less than 2%. The vials were stoppered and sealed.

The obtained Pemetrexed disodium was then used to prepare stable ready-to-use pharmaceutical composition, wherein the parenteral formulation as provided may be prepared and presented as a Single Vial formulation. The pharmaceutical composition was prepared by purging nitrogen into water for injection until dissolved oxygen content of water for injection comes to less than less than 7 mg/L, preferably less than 3 mg/L. Prepared Pemetrexed disodium was then added and stirred in water for injection. The pH of bulk solution was adjusted in between 6.6 to 7.8 with 10% w/v sodium hydroxide solution. Continued stirring was done to dissolve the Pemetrexed disodium. Final volume was made upto 100% with water for injection and drug solution was then filtered through a suitable 0.2μ filter. Nitrogen purging was continued throughout the entire process. Filtered solution was then filled into vials and vial headspace was blanketed with nitrogen to achieve headspace oxygen content less than 8%, preferably less than 2%. The vials were then stoppered and sealed. The unit Composition Formula of the pharmaceutical composition prepared by the present inventors is provided in Table-A:

TABLE A
Unit Composition Formula of the Stable ready-to-use formulation of
Pemetrexed

| Sr. No. | Ingredients | Qty/mL |
|---|---|---|
| 1 | Pemetrexed Diacid | 25 mg |
| 2 | Sodium Hydroxide/Hydrochloric acid | qs to pH 6.6-7.8 |
| 3 | Water for Injection | qs to 1 mL |
| 4 | Nitrogen* | qs |
| 5 | Antimicrobial agent** | |

*Nitrogen is used for purging in bulk solution and blanketing in vial headspace
**Antimicrobial agent is used to increase the storage stability and may be added optionally.

The ready-to-use formulation of Pemetrexed as presented in Table-A and thus prepared by the abovementioned process does not contain any antioxidants or amino acids or chelating agents in the formulation and yet exhibits comparable stability profile with that of the currently available marketed formulation of Pemetrexed.

Stability Data of the Pharmaceutical Composition of the Present Invention and its Comparison with the Stability Data of the Innovator Alimta® (Pemetrexed for Injection, M/s. Eli Lilly)

Stability of the pharmaceutical composition of the present invention was tested at initial stage and by subjecting the samples under various storage conditions: 40° C./75° % RH for 1 month and 2 month, 25° C./60% RH for 3 months and 6 months and 2-8° C. for 3 months and 6 months. Impurity analysis of formulation was done during initial stage and after storage under various conditions for various time periods. Samples of commercially available lyophilized product Alimta® was also analyzed at initial stage, 40° C./75% RH for 1 month 3 month and 6 month, 25° C./60° % RH for upto 6 months. The stability data of pharmaceutical composition of present invention and commercially available formulation are presented in Table 01 and 02 respectively. Table 03 shows the comparative stability data of pharmaceutical composition of the present invention and marketed formulation Alimta®.

TABLE 01
Stability Data of the ready-to-use formulation of Pemetrexed of the
present Invention

| Station | pH | Assay (%) | Total Related substance |
|---|---|---|---|
| Initial | 7.52 | 101.8 | 0.30 |
| 40° C./75% RH 1 Month | 7.68 | 102.2 | 0.50 |
| 40° C./75% RH 2 Month | 7.72 | 102.6 | 0.87 |
| 25° C./60% RH 3 Month | 7.25 | 97.9 | 0.44 |
| 25° C./60% RH 6 Month | 7.50 | 101.1 | 0.65 |
| 2°-8° C. 3 Month | 7.57 | 96.7 | 0.37 |
| 2°-8° C. 6 Month | 7.54 | 101.7 | 0.45 |

TABLE 02
Stability Data of Marketed ALIMTA ® Lyophilized Formulation

| Station | pH | Assay (%) | Total Related substance |
|---|---|---|---|
| Initial | 7.16 | 104.75 | 0.16 |
| 40° C./75% RH 1 Month | — | — | 0.23 |
| 40° C./75% RH 3 Month | — | — | 0.25 |
| 40° C./75% RH 6 Month | — | — | 0.34 |
| 25° C./60% RH 6 Month | — | — | 0.23 |

TABLE 03
Stability data of the pharmaceutical composition of the present invention and its comparison
with the stability data of the Marketed Alimta ® (LyophilizedFormulation, M/s. Eli Lilly)
Prior to any comparison it should be kept in mind that exact comparison of the stability
data cannot be done for the ready-to-use solution formulation of Pemetrexed of the
present Invention and Marketed ALIMTA ® Lyophilized Formulation, the reason being
that the pharmaceutical composition of the present invention is a ready-to-use solution
formulation, whereas Alimta ® is a lyophilized formulation. Thus both the dosage forms
are different from each other and storage stability conditions for both the compositions
are different. For example normal storage condition in case of the ready-to-use solution
formulation is at refrigerated temperature i.e. 2-8° C. and therefore, accelerated stability
testing is established at 25° C./60% RH. However, in the case of Lyophilized formulation
such as Alimta ®, storage condition is at room temperature i.e. at 25° C. and Accelerated
stability testing is established at 40° C./75% RH.
Therefore considering this, below mentioned is the comparison between ALIMTA ®
Lyophilized Formulation and the ready-to-use solution formulation of Pemetrexed of the
present Invention at initial stage and at various storage conditions.

| | | | |
|---|---|---|---|
| Station | pH | Assay (%) | Total Related substance |

Ready-to-use solution formulation of Pemetrexed of the present Invention

| | | | |
|---|---|---|---|
| Initial | 7.52 | 101.8 | 0.30 |
| 25° C./60% RH 3 Month | 7.25 | 97.9 | 0.44 |
| 25° C./60% RH 6 Month | 7.50 | 101.1 | 0.65 |
| 2°-8° C. 3 Month | 7.57 | 96.7 | 0.37 |
| 2°-8° C. 6 Month | 7.54 | 101.7 | 0.45 |

ALIMTA ® Lyophilized Formulation

| | | | |
|---|---|---|---|
| Initial | 7.16 | 104.75 | 0.16 |
| 40° C./75% RH 1 | — | — | 0.23 |
| Month | | | |
| 40° C./75% RH 3 | — | — | 0.25 |
| Month | | | |
| 40° C./75% RH 6 | — | — | 0.34 |
| Month | | | |
| 25° C./60% RH 6 | — | — | 0.23 |
| Month | | | |

From the stability data provided in the abovementioned Tables 01, 02 and 03, it may be mentioned that the ready-to-use solution formulation of Pemetrexed of the present Invention was found to be stable at various storage conditions: i.e at normal storage conditions at 2-8° C. for 3 months and 6 months, at accelerated storage conditions at 25° C./60° % RH for 3 months and 6 months and further at 40° C./75% RH for 1 month and 2 months and the impurity levels were also found to be under control during this time. Also the stability results at initial stages, normal storage conditions and also under accelerated storage conditions were found to be comparable with the stability data of commercially available ALIMTA® Lyophilized Formulation.

Further as mentioned above, the stability of the pharmaceutical composition of the present invention has been achieved by controlling the total oxygen content in the drug solution and vial headspace with the use of Nitrogen. The effect of controlling the oxygen content in the ready-to-use solution formulation of Pemetrexed of the present Invention was evaluated by comparing it to a formulation where no nitrogen was used to control the oxygen content of the formulation. Both the formulations were analyzed at initial stage and after subjecting them to storage for 14 days at 40° C./75° % RH. The stability data obtained is presented in Table 04.

TABLE 04
Stability data comparison of the Pharmaceutical composition of the
ready-to-use solution formulation of Pemetrexed of the present
Invention and Pharmaceutical composition without using nitrogen

| | | | |
|---|---|---|---|
| Conditions | Assay (%) | pH | Total Related Substance |

Ready-to-use solution formulation of Pemetrexed of the present Invention
(Using Nitrogen)

| | | | |
|---|---|---|---|
| Initial | 101.6 | 7.6 | 0.3 |
| 40° C./75% RH/14 days | 101.1 | 7.9 | 0.3 |

Pharmaceutical composition without using nitrogen

| | | | |
|---|---|---|---|
| Initial | 103.2 | 7.3 | 0.5 |
| 40° C./75% RH/14 days | 91.1 | — | 8.6 |

Hence, as seen from Table-04, at 14 days 40° C./75° % RH accelerated stability storage condition, in the pharmaceutical composition without using nitrogen, a significant fall in assay and very high level of impurities were observed, whereas in the pharmaceutical composition of the present invention at same storage condition no such fall in assay or development of impurities was observed. Hence the data presented in Table 04 demonstrates the stabilization of the ready-to-use solution formulation of Pemetrexed of the present Invention by controlling the oxygen content in the pharmaceutical and headspace with the use of Nitrogen.

As mentioned above and also as seen from the data provided in Table 04, the stability of the pharmaceutical composition of the present invention has been achieved by controlling the total oxygen content in the drug solution and vial headspace with the use of Nitrogen. The effect of controlling the oxygen content by using Nitrogen in the ready-to-use solution formulation of Pemetrexed of the present Invention was evaluated by comparing it to a formulation where Nitrogen was not used to control the oxygen content in the formulation and in its place antioxidants were used to control the oxygen content of the formulation. Both the formulations were analyzed at initial stage and after subjecting them to one month of storage at 40° C./75% RH. The stability data obtained is presented in Table 05.

TABLE 05
Stability data comparison of the Pharmaceutical composition of the
ready-to-use solution formulation of Pemetrexed of the present Invention
and Pharmaceutical composition using Antioxidants in place of Nitrogen

| | | | |
|---|---|---|---|
| Station | pH | Assay (%) | Total Related substance |

Pharmaceutical composition of the ready-to-use solution formulation of
Pemetrexed of the present Invention (using Nitrogen)

| | | | |
|---|---|---|---|
| Initial | 7.52 | 101.8 | 0.30 |
| 40° C./75% RH 1Month | 7.68 | 102.2 | 0.50 |

Pharmaceutical composition using Antioxidants in place of Nitrogen

| | | | |
|---|---|---|---|
| Initial | 7.25 | 100.12 | 3.10 |
| 40° C./75% RH 1 Month | 6.76 | 72.52 | 20.13 |

As seen from Table-05, it may be mentioned that results of the pharmaceutical composition of the ready-to-use solution formulation of Pemetrexed of the present Invention (using Nitrogen) at the initial time points and after subjecting them to 1 month at 40° C./75% RH were found to be more stable in comparison to the Pharmaceutical composition using Antioxidants in place of Nitrogen under the same storage conditions.

Hence, as seen above, the pharmaceutical composition of the ready-to-use solution formulation of Pemetrexed of the present Invention as illustrated in the abovementioned example is free of antioxidants or amino acids or chelating agents and is found to exhibit comparable stability profile to the currently marketed ALIMTA® lyophilized formulation and found to more stable in comparison to the Pharmaceutical composition using Antioxidants in place of Nitrogen under the same storage conditions.

1. A stable ready-to-use pharmaceutical composition comprising pemetrexed or a pharmaceutically acceptable salt thereof, wherein the composition is free from antioxidants, amino acids and chelating agents.

2. The pharmaceutical composition of claim 1, wherein the composition has a dissolved oxygen content of less than 7 mg/L.

3. The pharmaceutical composition claim 1, wherein pemetrexed is present at a concentration of about 2.5 to about 50 mg/ml.

4. The pharmaceutical composition of claim 1, further comprising a parenterally acceptable aqueous solvent.

5. (canceled)

6. The pharmaceutical composition of claim 1, having a pH in the range of 4 to 9.

7-8. (canceled)

9. The pharmaceutical composition of claim 1, wherein the composition further comprises a pH adjusting agent.

10. (canceled)

11. The pharmaceutical composition of claim 1, further comprising at least one antimicrobial agent.

12. The pharmaceutical composition of claim 11, wherein the antimicrobial agent is selected from the group consisting of benzalkonium chloride, benzyl alcohol, phenol, chlorocresol, phenylmercuric salts, methylhydroxybenzoate, propylhydroxybenzoate.

13. (canceled)

14. The pharmaceutical composition of claim 1, wherein the composition is provided in a sealed vial with a gaseous headspace, the headspace having an oxygen content of less than 8% by volume.

15. A ready-to-use pharmaceutical composition consisting of pemetrexed or a pharmaceutically acceptable salt thereof, water for injection, at least one pH adjusting agent and optionally at least one antimicrobial agent, wherein the composition has a dissolved oxygen content of less than 7 mg/L.

16-19. (canceled)

20. A process for preparing the stable ready-to-use pharmaceutical composition of claim 1, comprising the steps of:
i) purging inert gas into a parenterally acceptable aqueous solvent until the dissolved oxygen content of the solvent comes to less than 7 mg/L,
ii) adding pemetrexed disodium under stirring,
iii) adjusting the pH of the resulting solution to between 4 to 9, and
iv) optionally adding additional aqueous solvent, wherein the composition is purged with inert gas throughout the entire process.

21. The process of claim 20, wherein pemetrexed disodium is produced by adding a predetermined quantity of sodium hydroxide to a slurry of pemetrexed diacid in a parenterally acceptable aqueous solvent while purging with inert gas.

22. The process of claim 20, wherein the composition is subsequently filtered through a 0.2 μm filter.

23. The process of claim 20, wherein the composition is subsequently filled into a vial, wherein the vial headspace is flushed with an inert gas to achieve a headspace oxygen content of less than 8%, followed by sealing the vials.

24. The process of claim 20, wherein the inert gas is selected from the group consisting of argon, helium and nitrogen, or mixtures thereof.

25. (canceled)

26. The process of claim 20, wherein in step iii) the pH is adjusted with a pH adjusting agent selected from hydrochloric acid and/or sodium hydroxide.

27. The process of claim 20, further comprising the addition of at least one antimicrobial agent.

28. (canceled)

29. A method of treating a subject suffering from malignant pleural mesothelioma, the method comprising administering the pharmaceutical composition of claim 1 to a subject in a need of such treatment.

30. A method of treating a subject suffering from refractory non-small cell lung cancer comprising administering the pharmaceutical composition of claim 1 to a subject in a need of such treatment.

31. The method of claim 29, wherein the pharmaceutical composition is administered parenterally.
